(19) United States
(12) Patent Application Publication
(10) Pub. No.: US 2008/0300391 A1
(43) Pub. Date: Dec. 4, 2008

(54) Acid Mediated Deacylation of 6-0-Trichlorogalactosurcrose to Trich-Lorogalactosucrose

(76) Inventors: Rakesh Ratnam, Karnataka (IN); Sundeep Aurora, Karnataka (IN); Batchu Chandrashekar, Karnataka (IN); Anandangowda Patil, Karnataka (IN)

Correspondence Address: NATH & ASSOCIATES, 112 South West Street, Alexandria, VA, 22314, US

(21) Appl. No.: 12/083,859

(22) PCT Filed: Oct. 17, 2006
(86) PCT No.: PCT/IN2006/000424

§ 371 Date: Jul. 2, 2008

(30) Foreign Application Priority Data
Oct. 20, 2005 (IN) 1326/MUM/2005

(51) Int. Cl. C07H 1/00

(52) U.S. Cl. 536/18.4

(57) ABSTRACT

A process is described for acid mediated deacylation of acyl derivatives of chlorinated sucrose compounds comprising generating a predominantly organic phase condition in a process stream requiring deacylation treatment but allowing an optimum quantity of water content which is capable of participating in a hydrolysis reaction; this objective being achieved for the said process stream by following steps of (a) adding to it alcoholic solvent in an amount such that water content of final reaction mixture is between about 5% to 0.5%, (b) adding acid chloride to the same (c) adjusting the pH to 4 by adding acetate buffer in a methanolic solution and, (d) stirring the reaction until deacylation is complete. This process can be integrated in a process for production of a chlorinated compound, involving use of dimethylformamide during the process, to achieve deacylation without decomposition of dimethylformamide as well as the chlorinated sucrose product.

### TECHNICAL FIELD

The present invention relates to acid mediated deacylation of 6-O-trichlorogalactosucrose (TGS) to TGS during the process of production of TGS (1′-6′-Dichloro-1′-6′-DI DEOXY-β-Fructofuranasyl-4-chloro-4-deoxy-galactopyranoside).

### BACKGROUND OF THE INVENTION

Strategies of prior art methods of production of 4,1′,6′ trichlorogalactosucrose (TGS) predominantly involve chlorination of sucrose-6-ester by use of Vilsmeier-Haack reagent derived from various chlorinating agents such as phosphorus oxychloride, oxalyl chloride, phosphorus pentachloride etc, and a tertiary amide such as dimethyl formamide (DMF) or dimethyl acetamide to chlorinate Sucrose-6-ester, to form 6 acetyl 4,1′,6′trichlorogalactosucrose. After the said chlorination reaction, the reaction mass is neutralized to pH 7.0-7.5 using appropriate alkali hydroxides of calcium, sodium, etc. The pH of the neutralized reaction mass is then further raised up to 9.0-9.5 to deesterify/deacetylate the 6 acetyl 4,1′,6′trichlorogalactosucrose to form 4,1′,6′ trichlorogalactosucrose (TGS).

All known prior art processes to deacylate the 6-O-protected TGS in a solution in an organic or and aqueous condition before and after isolation of the said compound use a base as the deacylating agent and the pH condition is maintained in the alkaline range, usually above 9.5 to 11.5. During the process of this deacylation, it has been noticed that TGS formed is not very stable at the said pH and starts to decompose. So it is mandatory to neutralize the mass immediately after deacylation in any of the said processes. Yet some loss is unavoidable. Also if the tertiary amide is present during the deacylation, its exposure to alkaline pH makes it vulnerable to hydrolysis to dialkyl amine and carboxylic acid. In the industrial process, this results in reduced recovery of the tertiary amide and affects the economics of the process.

An alternative process is needed which shall avoid loss of TGS as well as DMF during the deacylation process.

### SUMMARY OF THE INVENTION

This invention describes a process to deacylate the 6-O-TGS under acidic conditions. Under acidic conditions a tertiary amide particularly OMF is stable and hence in reaction mixtures containing DMF, this reaction will carry out deacylation without destruction of any DMF. The process involves (a) creating predominantly organic phase in the reaction mixture but yet containing water sufficient to participate in a hydrolysis reaction; by maintaining when water content of the reaction mixture to a low level, preferably at or below 5% but above about 0.5% (b) adding an alcoholic solvent including but not limited to methanol, ethanol, butanol and the like in a preferable V/V proportion of reaction mixture to alcoholic solvent as 1:1 or above; (c) acidifying, preferably by adding acyl halides such as acetyl chloride, acetyl bromide, Propionyl chloride, Oxalyl chloride, Chloroacetyl chloride and the like (d) adjusting the pH to about 4 preferably aided by addition of a buffer, preferably an acetate buffer in an alcoholic solvent and (e) stirring the reaction mixture until deacylation is over.

This deacylation is achieved in a reaction mixture containing 6-O-TGS as well as in a solution containing the same purified at various extent and stages. In a reaction mixture containing DMF, this method of deacylation gives an advantage that there is practically no decomposition of DMF as well as TGS during deacylation, as contrasted to significant loss of deacylation of the both during conventional process of deacylation under alkaline conditions.

### DETAILED DESCRIPTION OF THE INVENTION

This invention discloses a process of deacylation of acyl derivatives/precursors of a chlorinated sucrose compound in acidic condition. The process is applicable to deacylate 6-O-protected chlorinated sucrose in a process of production of the high intensity sweetener TGS. It has been found that hydrolysis of acyl derivative of chlorinated sucrose is possible under acidic conditions, around pH 4 in presence of low to trace amounts of water and an alcoholic solvent and it was surprising to note that in such conditions there is no destruction of TGS formed as well as that of DMF.

Mechanism of acid mediated deacylation is likely to be following: One mole of an acid chloride is required for deacylation of 1 mole of TGS-6-acetate wherein the acid chloride reacts with the methanol, HCl is liberated. The liberated HCl cleaves the acyl group in TGS-6-acetate to form TGS. This also requires presence of water in traces for the reaction to happen but in trace amounts.

In actual practice, a water content of about 5% to 0.5% of the final volume of reaction mixture and addition of methanol are found to be critical factors besides maintaining pH to 4 for satisfactory acid deacylation.

A reaction containing TGS-6-acetate when subjected to direct acid hydrolysis in the presence of water up to 5-0.5% but in absence of methanol results in incomplete deacylation. The reason is the reaction requires an alcoholic solvent, which facilitates protonation.

The experiment with excess water above 5% results in incomplete or slow deacylation. Also at acidic pH under aqueous conditions, the TGS formed will degrade due to the breakage of the glycosidic link.

In this process, volume of alcoholic solvent required shall get reduced to the extent to which water production/addition during various steps of the process prior to the addition of the said alcoholic solvents could be restricted. This is achieved by use of ammonia gas for neutralization of the chlorination reaction mixture, which again is one of the embodiments of this invention, instead of conventional process of using solution/slurry of alkali hydroxides.

The acid mediated deacylation of 6-O-TGS can be performed during any part of the extraction and purification process of TGS from the chlorinated mass. It can be used,
a) After neutralization of the chlorinated mass.
b) As a crude compound along with other chlorinated sucrose derivatives at intermediate stage of TGS isolation.
c) After complete isolation of 6-O-TGS.

In the embodiment (a), the chlorination of the 6-acyl sucrose is carried out by first reacting the chlorinating agents such as Phosphorus oxychloride, phosphorus pentachloride, Triphosgene, etc., with DMF to form the Vilsmeier-Haack reagent. The solution is then cooled to −5 to 10° C. more preferably between 0-5° C. and Ore 6-acyl sucrose dissolved in DMF is added dropwise with constant stirring. After the addition of the 6-acyl sucrose solution, the reaction mass is slowly allowed to attain room temperature and stirred for 60 minutes. Then the mass is heated to elevated temperature to 80-90° C. preferably to 85-87° C. and held for 60 minutes and further heated to 90-110° C. preferably to 100° C. and held for 5-6 hours and finally held for 90 minutes at 115° C. The chlorinated reaction mass is then neutralized under anhydrous conditions by bubbling ammonia gas in to the reaction mass till the pH of the reaction mass reached 6-8 preferably at 7.0-7.5. The deacylation can be carried out at this stage wherein 1:1 to 1:3 v/v of methanol is added to the reaction mass and an acid halide such as acetyl chloride, acetyl bromide, Propionyl chloride, Oxalyl chloride is added and the pH is controlled at preferably 3.5 to 4.0 using an appropriate buffer solution. The reaction was stirred continuously for 3-24 hours more preferably 15-20 hrs still more preferably 5-10 hrs wherein the deacylation was completed. Embodiments of a chlorination reaction mixture to which this invention can be applied includes typically every process of production of chlorinated sucrose wherein an acylated chlorinated sucrose compound needs to be deacylated. Examples of such situations include a process stream obtained as a chlorination reaction mass as descried by, including but not limited to, one or more of following: as described in Mufti et al. (1983) U.S. Pat. No. 4,380,476, Walkup et al. (1990 U.S. Pat. No. 4,980,463), Jenner et al. (1982) U.S. Pat. No. 4,362,869, Tulley et al. (1989) U.S. Pat. No. 4,801,700, Rathbone et al. (1989) U.S. Pat. No. 4,826,962, Bornemann et al. (1992) U.S. Pat. No. 5,141,860, Navia et al. (1996) U.S. Pat. No. 5,498,709, Simpson (1989) U.S. Pat. No. 4,889,928, Navia (1990) U.S. Pat. No. 4,950,746, Neiditch et al. (1991) U.S. Pat. No. 5,023,329, Walkup et al. (1992) U.S. Pat. No. 5,089,608, Dordick et al. (1992) U.S. Pat. No. 5,128,248, Khan et al. (1995) U.S. Pat. No. 5,440,026, Palmer et al. (1995) U.S. Pat. No. 5,445,951, Sankey et al. (1995) U.S. Pat. No. 5,449,772, Sankey et al. (1995) U.S. Pat. No. 5,470,969, Navia et al. (1996) U.S. Pat. No. 5,498,709, Navia et al. (1996) U.S. Pat. No. 5,530,106.

In embodiment (b), the neutralized mass containing 6-acyl TGS was extracted in to 1:3 times of water immiscible solvent such as ethyl acetate, butyl acetate, dichloromethane, etc and the extract was concentrated to 50% of its initial volume. The DMF which is co-extracted along with 6-acetyl TGS into the solvent was removed by washing the organic extract with saturated sodium chloride solution for a number of times till the DMF in the organic extract was less than 0.1-0.5%. Then after complete concentration of the organic layer, diluting the syrup obtained with a suitable alcoholic solvent and the addition of an acid halide to carry out the acid mediated deacylation.

In the embodiment (c), the 6-acetyl TGS after the removal of the organic solvent after DMF removal by saturated sodium chloride washing was subjected to further purification by column chromatography, etc. to obtain a pure fraction of 6-acetyl TGS. This fraction can be concentrated and extracted into water immiscible solvents such as ethyl acetate, butyl acetate, dichloromethane, etc and the subjected to concentration and the pure 6-acetyl TGS obtained free from water can be subjected to acid mediated deacylation by addition of an appropriate alcohol and the acid halide.

Further, besides 6-O-TGS, one or more of an acyl derivative of a chlorinated sucrose compound can be deacylated by this process including but not limited to different dichloro and tetrachloro derivatives, 4,1′ dichlorosucrose-6-acetate, 1′6′ dichlorosucrose-6-acetate, 4,1′,5′-trichlorogalactosucrose-6-acetate and the like.

Described in the following are examples, which illustrate working of this invention without limiting the scope of this invention in any manner. Reactants, proportion of reactants used, range of reaction conditions described are only illustrative and the scope of this invention extends to their analogous reactants, reaction conditions and reactions of analogous generic nature. In general, any equivalent alternative, which is obvious to a person skilled in art of chlorinated sucrose production is covered within the scope of this specification. Mention in singular is construed to cover its plural also, including all equivalent alternatives encompassed by that expression, unless the context does not permit so, viz: use of “a chlorinated sucrose” includes all chlorinated sucrose compounds individually as well as mixtures thereof or an alternative chlorinated sucrose compound that may perform same function in a relevant context. A mention of “an organic solvent” for solution covers use of one or more of an organic solvent in succession or in a combination as a mixture or any one of the several alternatives capable of performing same function as claimed, described in the description or illustrated in one or more of an example.

### EXAMPLE 1

### Chlorination of 6-acetyl Sucrose by a Prior Art Method

25.2 g of PCl5 was added to 320 ml of DMF taken in a reaction flask under constant stirring at temperature between 30-35° C. The reaction mass was stirred for 30 minutes for the Vilsmeier to be formed and then the contents were cooled to 0° C. 63 g of 6-acetyl sucrose solution in DMF was added dropwise to the reaction flask and the temperature was maintained below 5° C. with stirring. After the addition, the temperature was allowed to RT and stirred for 60 minutes. The temperature was then increased to 85° C. maintained for 60 minutes, heated again to 100° C., maintained for 6 hours and further heated to 115° C. and maintained for 2 hrs. The reaction mass was then neutralized using ammonia gas by bubbling the gas through the reaction mixture till the pH was 7.0.

### EXAMPLE 2

### Acid Mediated Deacylation of Neutralized Mass

250 ml of the neutralized mass from example 1 was taken for deacylation. 250 ml containing 70 g of 6-O-protected TGS was mixed with 250 ml of methanol. 12 ml of acetyl chloride was added dropwise to the reaction flask kept under stirring.

The temperature was controlled below 35° C. After the addition of the acetyl chloride, the pH was adjusted to 4.0 using acetate buffer prepared in methanolic solution. The reaction was kept stirring and TLC was checked every one hour to monitor deacylation.

At the end of 5 hours, the TLC showed absence of 6-O-TGS and the conversion to TGS. Complete deacylation was confirmed by HPLC. The overall yield loss during deacylation was less than 0.05%. The loss of DMF during the deacylation was found to be nil.

### EXAMPLE 3

### Chlorination and Deacylation by Prior Art Method

25.2 g of PCl5 was added to 320 ml of DMF taken in a reaction flask under constant stirring at temperature between 30-35° C. The reaction mass was stirred for 30 minutes for the Vilsmeier to be formed and then the contents were cooled to 0° C. 63 g of 6-acetyl sucrose solution in DMF was added dropwise to the reaction flask and the temperature was maintained below 5° C. with stirring. After the addition, the temperature was allowed to RT and stirred for 60 minutes. The temperature was then increased to 85° C. maintained for 60 minutes, heated again to 100° C., maintained for 6 hours and further heated to 115° C. and maintained for 2 hrs. The reaction mass was then neutralized using ammonia gas by bubbling the gas through the reaction mixture till the pH was 7.0.

250 ml of the neutralized mass was taken for deacylation. Calcium hydroxide slurry in water was added to the mass and the pH was adjusted to 9.5 and was stirred for 8 hours. The deacetylation of 6-acetyl TGS to TGS was monitored by TLC system every hour. After the completion of the deacetylation, the TGS loss and the DMF loss was found to be 3.5% and 8% respectively.

### EXAMPLE 4

### Acid Mediated Deacylation After Ethyl Acetate Extraction of Neutralized Mass

8.0 L of neutralized mass generated after chlorination by prior art method described in Example 1 was taken for the experiment. The 6-O-TGS (280 g) in the neutralized mass was extracted into 1:3.0 times v/v of ethyl acetate, The layers were then allowed to separate and the organic layer was concentrated up to 50% of its original volume and was washed with 1:0.1 v/v of saturated sodium chloride for 8 times till the DMF was reduced to less than 0.5% in the solution. Then the ethyl acetate layer was concentrated completely and syrup was obtained.

The concentrate was diluted with 1:2 times w/v of methanol and stirred well. 65 ml of Acetyl chloride was added slowly dropwise to the mixture and the temperature was maintained below 40° C. After the addition of the acetyl chloride, the pH of the reaction mass was controlled to 4.0 by addition of acetate buffer. The reaction was stirred continuously and the deacylation was monitored by TLC. The deacylation time taken was 16 hrs.

After the deacylation, the methanol was stripped off by distillation and the reaction mass was neutralized and taken for TGS isolation.

The pH of the reaction mass was adjusted to neutral using 20% NaOH solution. The syrup was then loaded on to silanized silica gel column and the mobile phase used was acetate buffer at pH 10.5. The pure TGS fractions were pooled together and concentrated.

The concentrate was then extracted into 1:3.5 times of ethyl acetate and was concentrated and crystallized. The overall yield of TGS obtained by the process was 28% of 6-acetyl sucrose input.

### EXAMPLE 5

### Acid Mediated Deacylation of Isolated 6-acetyl TGS

12.0 L of neutralized mass generated after chlorination by prior art method described in Example 1 was taken for the experiment. The 6-acetyl TGS content in the mass was 2.43% w/v.

The 6-acetyl TGS was then extracted into 1:3 times of ethyl acetate and was subjected to 50% concentration. The ethyl acetate extract was then washed with 1:0.1 times v/v of saturated sodium chloride solution to remove the DMF and was repeated 10 times.

The ethyl acetate was then completely removed and a syrup was obtained which was loaded on to a silanized silica gel column. The separation was carried out by using acetate buffer at pH 10.5. The pure fractions of 6-acetyl TGS was then concentrated and extracted with 1:3.5 times of ethyl acetate. The ethyl acetate extract was then concentrated completely and taken for deacylation.

500 ml containing 30 g of 6-O-protected TGS was mixed with 500 ml of methanol. 7 ml of acetyl chloride was added dropwise to the reaction flask kept under stirring. The temperature was controlled below 35° C. After the addition of the acetyl chloride, the pH was adjusted to 3.5 using acetate buffer prepared in methanolic solution. The reaction was kept stirring and TLC was checked every one hour to monitor deacylation.

At the end of 12 hours, the TLC showed absence of 6-O-TGS and the conversion to TGS. Complete deacylation was confirmed by HPLC.

The Deacetylated product was then subjected to methanol removal and then TGS was crystallized. The purity was found to be 96.8%.

1. A process for production of a chlorinated sucrose compound comprising a step of deacylation of a solution of an acyl-derivative of the said chlorinated sucrose compound wherein the said step of deacylation is achieved under an acidic condition in presence of an alcoholic solvent and in a quantity of water enough for hydrolysis but low enough to prevent acid hydrolysis of TGS.

2. A process of claim 1 wherein:
a. the said solution is a solution at neutral pH, preferably around pH 7, of partly pure or a substantially pure solution of an acylated chlorinated sucrose compound dissolved in a liquid or is a process stream obtained in a process of production of a chlorinated sucrose comprising chlorination of acyl-derivative of the chlorinated sucrose compound,
b. the acyl derivative of the said chlorinated sucrose compound comprises one or more of mono chloro, dichloro and tetrachloro derivative and the like,
c. adding an alcoholic solvent to the said solution in an amount enough to achieve water content of the final reaction mixture remains low, preferably within a range of 5% to 0.5%,
d. achieving an acidic condition, preferably by adding an acidifying agent further preferably generating an acid after its addition by reaction with alcoholic solvent present in the process stream at the end of step (c), the said step of generating acid being preferably achieved by addition of an acid halide or the like,
e. maintaining pH to acidic range preferably to around 4 further preferably very close to 4 by adding a buffer, and
f. keeping the process stream under stirring conditions for a period of time enough for complete deacylation.

3. A process of claim 3 wherein:
a. the said chlorinated sucrose compound comprises one or more of, 4-monochlorosucrose-6-acetate, 1′monochlorosucrose-6-acetate, 6′monochlorosucrose-6-acetate, 4,1′ dichlorosucrose-6-acetate, 1′6′dichlorosucrose-6-acetate, 4,1′,5′-trichlorogalactosucrose-6-acetate, 4,1′,5′,6′tetrachlorogalactosucrose-6-acetate and the like,
b. the said alcoholic solvent comprising one or more of methanol, ethanol, butanol and the like,
c. the said acid halide comprises one or more of acetyl chloride, acetyl bromide, Propionyl chloride, Oxalyl chloride, Chloroacetyl chloride and the like,
d. the said buffer is preferably an acetate buffer of pH 4.

4. A process of production of 4,1′,6′ trichlorogalactosucrose comprising one or more of a step of:
a. Obtaining:
i: a reaction mixture as a process stream of a process of production of 4,1′,6′ trichlorogalactosucrose, the said process stream comprising one or more of 6-O-acetyl, 4,1′6′ trichlorogalactosucrose, inorganic salts, related organic impurities, caramelization products, dimethylformamide, and the like,
ii. neutralizing the said process stream with an alkali, preferably by ammonia gas to obtain a process stream and subjecting the process stream to step (b), or,
iii. subjecting the said process stream of step (ii) to extraction of 6-O-acetyl 4,1′6′trichlorogalactosucrose in an organic solvent, preferably in ethyl acetate, preferably concentrating the organic solvent extract further preferably to about 50% of its original volume, removing tertiary amide particularly dimethylformamide if present in the process stream by washing the organic solvent extract with saturated salt solution a number of times preferably about 10 times until dimethylformamide is substantially reduced preferably to about 0.5% in the organic solvent extract fraction, evaporating the organic solvent completely to get an essentially solvent free syrup, adding methanol preferably up to five times volume of the original weight of the syrup, and subjecting this process stream to step (b), or
iv. loading the said essentially solvent free syrup of step (iii) on a chromatographic column to separate 6-acetyl-4,1′6′ trichlorogalactosucrose preferably on a silanized silica gel column, carrying out separation by using acetate buffer in an alkaline pH preferably at a preferred pH of 10.5, concentrating the pure fractions of 6-acetyl 4,1′6′ trichlorogalactosucrose, extracting with a solvent preferably by about 1:3.5 times of ethyl acetate, concentrating by removing the ethyl acetate completely to obtain a syrup, adding equal volume of methanol to the syrup and subjecting this process stream to step (b),
b. adding to the said process stream obtained at the end of a step (ii), or (iii) or (iv) an acidifying agent preferably an acid halide further preferably acetyl chloride, preferably maintaining temperature to below 400 Celcius,
c. adjusting the pH as close as possible to 4 preferably by addition of a buffer further preferably an acetate buffer preferably prepared in an alcoholic solution further preferably comprising a methanolic solution, and
d. the reaction is kept stirred for a period of time till deacylation is complete,
e. the alcoholic solvent is stripped off by distillation, and
f. the solution is taken for 4,1′,6′ trichlorogalactosucrose isolation.
